(19) United States
(12) Patent Application Publication
(10) Pub. No.: US 2012/0237613 A1
(43) Pub. Date: Sep. 20, 2012

(54) FACE CREAM

(76) Inventor: Haedeh MOAZAMI, Potomac, MD (US)

(21) Appl. No.: 13/047,705

(22) Filed: Mar. 14, 2011

(51) Int. Cl. A61K 8/97; A61Q 19/00; A61Q 19/08

(52) U.S. Cl. 424/642

(57) ABSTRACT

A face and neck cream comprised of Zinc Oxide, Virgin Olive Oil, Coconut oil, Vaseline, and 100% Pure Essential Lemon Oil, and made by mixing, placing in the composition in the microwave oven for 1.5 minutes, allowing the composition to cool for one hour, placing the composition back in the microwave oven for another 1.5 minutes, then allowing the composition to cool to allow for a thicker consistency.

### CROSS-REFERENCE TO RELATED APPLICATION

None

### FEDERALLY SPONSORED RESEARCH

None

### SEQUENCE LISTING OR PROGRAM

None

STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND

The invention relates to a face cream composition and method of making said composition.

Face creams are known. For example, U.S. Pat. No. 5,705,166 to Arve consists of an Exfoliating Skin Cream comprised of a defoliating enzyme, namely, unripened papaya juice containing Papain and other substances occurring naturally in unripened papaya, mixed with a soothing ingredient, namely, honey, and concentrated to form a cream for topical application to skin tissue which is activated by an activating pH level. Both the composition and method making the composition are claimed.

Also, USP Publication No. US 2006/0134046 A1 by Hanna et al. is a super-moisturizing and anti-aging cream comprised of a water in oil emulsion blended to a creamy texture. The cream is produced by blending specific quantities of spermaceti, anhydrous lanolin, pure beeswax, sweet almond oil and coconut oil with glycerin, rose water, hydrolysed collagen, hyaluronic acid and distilled water at a certain temperature, then mixing at high speed.

The present invention consists of a different composition and method for making said composition for a face cream. The inventive face cream has been tested for close to a year. It has been found to not only moisturize the face and neck, but also helps to prevent signs of aging, hydrates the skin for a firmer appearance, minimizes the appearance of fine wrinkles, and restores a youthful radiance, giving the skin a healthy glow.

### SUMMARY

A face and neck cream comprised of Zinc Oxide, Virgin Olive Oil, Coconut oil, Vaseline, and 100% Pure Essential Lemon Oil, and made by mixing, placing in the composition in the microwave oven for 1.5 minutes, allowing the composition to cool for one hour, placing the composition back in the microwave oven for another 1.5 minutes, then allowing the composition to cool to allow for a thicker consistency.

### DRAWINGS

FIG. 1 shows the preferred embodiment of the face cream composition and method for making the composition.

### DETAILED DESCRIPTION

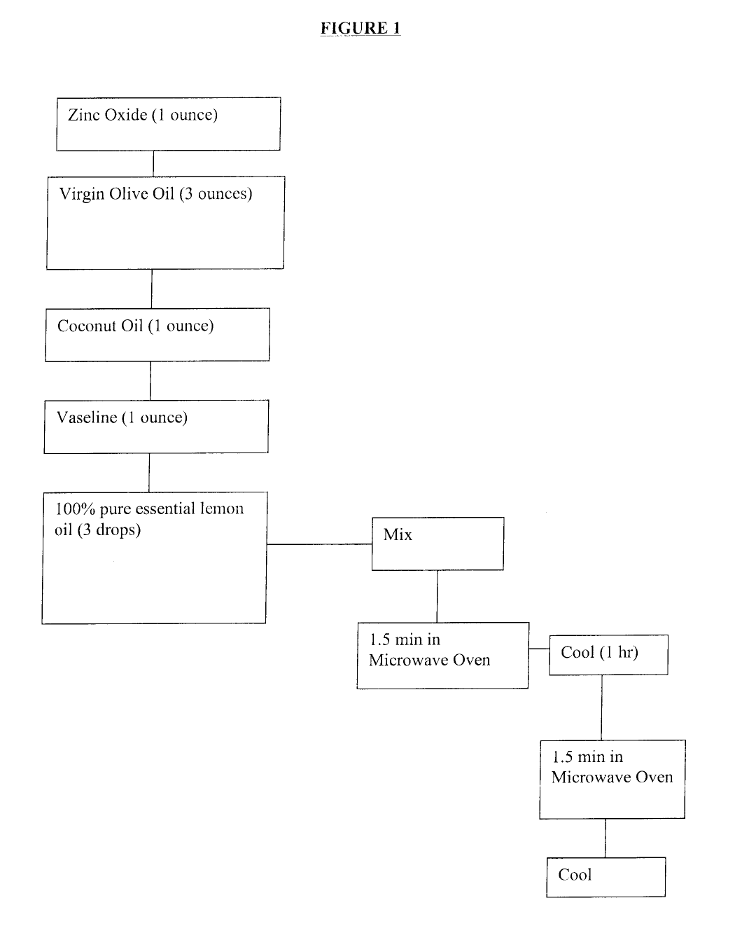

FIG. 1 illustrates the following composition and method of making said composition. Said composition consists of Zinc Oxide, Virgin Olive Oil, Coconut oil, Vaseline, and 100% Pure Essential Lemon Oil. These ingredients are mixed together, placed in a microwave, cooled, placed in a microwave oven again, and cooled again so that the consistency thickens.

The preferred embodiment of the composition is as follows:
Zinc Oxide (1 ounce)
Virgin Olive Oil (3 ounces)
Coconut Oil (1 ounce)
Vaseline (1 ounce)
100% Pure Essential lemon oil (3 drops)

The preferred method of making the composition is as follows:
Mix the composition
Place the composition in the microwave oven for 1.5 minutes set on high Cool the composition for one hour
Place the cooled composition in the microwave oven again for 1.5 minutes set on high
Allow the composition to cool again.

While the above constitutes the preferred embodiment of the composition and method of making the composition, the method and amount of the ingredients may vary as follows:
Zinc oxide: 1-4 ounces (1 ounce typically yields the best results for normal skin types)
Virgin Olive Oil: 2.5-6 ounces
Coconut oil: 0.5-4 ounces
Vaseline: 0.5-4 ounces
100% pure essential lemon oil: 2-8 drops

Moreover, virgin olive oil may be replaced by any other vegetable oil of similar consistency and light aroma in the same quantities. Examples include, but are not limited to, flaxseed oil and grape seed oil.

Other fruit extracts may be used in addition to, or in lieu of lemon oil drops, as long as they do not cause skin irritation or adversely affect the aroma of the product.

Vaseline may be replaced by another skin lubricant, such as glycerin, as long as it does not adversely affect the consistency of the composition (i.e., does not significantly reduce the viscosity of the overall composition).

All features disclosed in this specification, including any accompanying claims, abstract, and drawings, may be replaced by alternative features serving the same, equivalent or similar purpose, unless expressly stated otherwise. Thus, unless expressly stated otherwise, each feature disclosed is one example only of a generic series of equivalent or similar features.

Although preferred embodiments of the present invention have been shown and described, various modifications and substitutions may be made thereto without departing from the spirit and scope of the invention. Accordingly, it is to be understood that the present invention has been described by way of illustration and not limitation.

The following are claimed:

1. A face cream composition comprised of Zinc oxide, Virgin olive oil, coconut oil, Vaseline, and 100% pure essential lemon drops.

2. The face cream composition of claim 1 comprised of the following quantities: Zinc oxide (1 to 4 ounces), Virgin Olive Oil (2.5 to 6 ounces), Coconut Oil (0.5 to 4 ounces), Vaseline (0.5 to 4 ounces), 100% pure essential lemon drops (2 to 8 drops).

3. The face cream composition of claim 1 comprised of the following quantities: Zinc oxide (1 ounce), Virgin Olive Oil (3 ounces), Coconut Oil (1 ounce), Vaseline (1 ounce), 100% pure essential lemon drops (3 drops).

4. The face cream composition of claim 2, wherein the Virgin olive oil is replaced by any other vegetable oil of similar consistency and light aroma and in the same quantities, including flaxseed oil or grape seed oil.

5. The face cream composition of claim 1, wherein the 100% pure essential lemon drops is replaced by other fruit extracts that do not cause skin irritation.

6. The face cream composition of claim 2, wherein the Vaseline is replaced by another skin lubricant that does not significantly reduce the viscosity of the overall composition, including glycerin.

7. A method of making the composition of claim 1, comprised of the following steps: mix the composition, place the composition in a microwave oven for at least 1 minute, allow the composition to cool for at least 45 minutes, place the composition in the microwave oven again for at least one minute, and let the composition cool.

8. The method of making the composition of claim 7, comprised of the following steps: mix the composition, place the composition in a microwave oven for 1.5 minutes, allow the composition to cool for 1 hour, place the composition in the microwave oven again for 1.5 minutes, and let the composition cool.
